(19) United States
(12) Patent Application Publication
(10) Pub. No.: US 2014/0354952 A1
(43) Pub. Date: Dec. 4, 2014

(54) SYSTEM FOR AN OBSERVATION OF AN EYE AND ITS SURROUNDING AREA

(71) Applicant: UNIVERSITE DE LIEGE, Angleur (BE)

(72) Inventors: Jacques Verly, Angleur (BE); Serge Habraken, Comblain-au-pont (BE); Jérôme Wertz, Polleur (BE)

(73) Assignee: UNIVERSITE DE LIEGE, Angleur (BE)

(21) Appl. No.: 14/373,476

(22) PCT Filed: Jan. 17, 2013
(86) PCT No.: PCT/EP2013/050864

§ 371 Date: Jul. 21, 2014

(30) Foreign Application Priority Data
Jan. 22, 2012 (EP) 12152027.4

(51) Int. Cl. A61B 3/00; A61B 3/14

(52) U.S. Cl. CPC A61B 3/0008; A61B 3/14
USPC 351/206; 351/221; 351/213

(57) ABSTRACT

Disclosed is a system and device for observation of an eye (E) comprising a light source (LSo) for illuminating the eye. Further, the system includes a diffraction grating as an optical relay (OR) for receiving the light reflected from the eye and for deflecting it towards a light sensor (LSe). The incident light ray (r inc) intersects the optical relay at an incidence angle (alphajnc) with the normal (n_OR) to the optical relay in an incidence plane (Pl_inc) formed by the incident light ray (r_inc) and the normal (n_OR). The deflected light ray (r def) forms a deflection angle (alpha def) with the normal (n_OR) in a deflection plane (Pl def) formed by the deflected light ray (r_def) and the normal (n_OR). The optical relay is positioned such that the incidence and the deflection angle and/or the incidence plane and the deflection plane are different from each other.

The present invention relates to a system and a device for an observation of an eye and its surrounding area and to the use of such system and device in applications thereof.

Systems for an observation of an eye are well known in the art. Such systems are used in a number of applications, such as eye-tracking, medical diagnosis, and drowsiness monitoring.

Drowsiness is a major cause of accidents of various types, often with grave consequences. For example, drowsiness is reported to cause 20 to 30% of all car accidents, and, in the USA, 100,000 road accidents per year with 1,000 deceased persons and 71,000 injured persons. In addition, 6 to 11% of the population is reported to suffer from excessive daytime sleepiness, constituting a permanent potential danger. Drowsiness while driving is clearly a major problem of public safety and security. It is thus not surprising that there is an increasing trend for authorities to launch campaigns to prevent drowsiness at the wheel, and by companies to develop drowsiness monitoring systems to equip cars and drivers.

In general, there are various approaches for the observation of an eye. In the case of drowsiness monitoring, there are two main types of systems. In the first type of systems, the system sends pulses of light towards the eye, senses the reflected light, and analyses the corresponding electrical signals. Such systems fall in the category of OptoOculoGraphy, abbreviated as OOG. In the second type of systems, the system captures images of the eye with an imaging sensor, typically a camera. Such systems fall in the category of PhotoOculoGraphy, abbreviated as POG. While OOG systems naturally illuminate the eye at each time a pulse is sent out, POG systems also tend to illuminate the eye in an active and controlled way to insure a constant illumination in all light-level conditions, including in darkness. OOG and POG systems generally use the infrared (IR) part of the electromagnetic spectrum for the simple reason that illuminating the eye in the IR does not interfere with the normal vision of the user, since the eye does not see in the IR. One should, however, be careful to respect all norms related to the IR illumination of the eye to avoid injuries to this organ.

An advantage of using POG systems and therefore images is that this allows the monitoring system to distinguish between important parts of the eye area such as a pupil, an iris, and eyelids.

Particularly, the use of POG systems provides the advantage to offer both spatial resolution and temporal resolution, whereas OOG systems provide only temporal resolution.

For both OOG and POG, one can further distinguish between systems that are mounted on the user, such as on the head, and systems that are remotely placed, such as on a dashboard. The systems mounted on the user have several advantages such as providing higher spatial resolution (because of the closer distance between the eye and the camera), not requiring that the face and the eye be detected and tracked from a distance), and remaining effective even if the wearer leaves the wheel, as is conceivable in a freight train in an isolated area of a large country.

US 2008/0030685 A1 by Fergason et al. discloses a system for monitoring eye movements through an optical observation of an eye. The disclosed system comprises an optical device having a light source configured for emitting light along a first path and a sensor positioned to receive light from a second path nearly coincident with the first path. A reflector is located within a lens and configured to reflect light emitted by the light source onto the eye and to reflect light reflected by the eye to the sensor.

The problem with such a system is that the light source must necessary illuminate the eye via the reflector or reflective surface. The light source cannot illuminate the eye directly.

Moreover, the system requires that the path of light sent to the eye and the path of light received from the eye must necessarily go via the reflector or reflective surface and must nearly coincide. This is unnecessarily restrictive and wasteful of energy, and may result in excessive light reaching the eye, with the danger of exposing it to excessive electromagnetic radiation, in particular in the infrared (IR) part of the electromagnetic spectrum.

In addition, the presence of some type of reflector in a lens generally results in the reflector being less transparent than the surrounding lens region and consequently visible within the field of vision of a user.

EP0821908 A1 by Sharp et al. discloses an eye detection system with a light source, a deflector, and a detector. However, the deflector used is not transparent to light in the visible part of the electromagnetic spectrum and, therefore, is not convenient when placed in the field of vision of the user. Moreover, in such system, the incident light ray passes through the deflector before hitting the eye.

US2007/109619 A1 by Eberl et al. discloses a system for an observation of an eye comprising a light source, an optical relay, and a light sensor but again the illuminating light passes through a mirror before hitting the eye.

Finally, a major problem that affects the systems of the art is the fact that the reflector (or reflective surface) is always—explicitly or implicitly—considered to be acting as a mirror. The terms “reflector”, “reflection”, and the like imply that the relation between any light ray incident on the reflective surface and the corresponding redirected ray is highly constrained by some basic laws of optics.

The first constraint from optics is that the incident ray, reflected ray, and normal to the reflective surface at a point of intersection of the incident ray with the reflective surface must be in the same plane. This can be stated in an equivalent way by saying that the plane defined by the incident ray and the normal must be the same as the plane defined by the reflected ray and the normal.

The second constraint from optics is that—within the plane containing the incident ray, the normal, and the reflected ray—the angle of incidence (defined as the customary angle between the incident ray and the normal) must be equal to the angle of reflection (defined as the customary angle between the reflected ray and the normal).

These two constraints from optics severely limit the freedom of configuration in positioning the various elements used for the system for an observation of an eye. Particularly, a light source, a reflector or reflective surface, a light sensor, and possibly a filter should be carefully positioned with respect to the eye, in a configuration that can itself be very tight and constrained, as will likely be the case if these elements must interact on a spectacles frame, which is furthermore typically articulated.

We have now found a system for an observation of an eye that overcomes the problems of the state of the art and that also provides useful properties to the reflective surface. The system can be implemented with light pulses (OOG systems) and with images (POG systems).

In accordance with the present invention, there is provided a system for an observation of an eye (E) comprising:
a light source (LSo) positioned with respect to the eye (E) and configured to emit an illuminating light ray (r_ill) towards the eye (E);
an optical relay (OR) positioned with respect to the eye (E) and configured to receive an incident light ray (r_inc) resulting from the interaction between the illuminating light ray (r_ill) and the eye (E), wherein:
   the incident light ray (r_inc) intersects the optical relay (OR) at an intersection point (P_int), and
   forms an incidence angle (alpha_inc) with the normal (n_OR) to the optical relay (OR) at the point (P_int) in an incidence plane (Pl_inc) formed by the incident light ray (r_inc) and the normal (n_OR);
a light sensor (LSe) positioned with respect to the optical relay (OR) and configured to receive a deflected light ray (r_def) resulting from the interaction between the incident light ray (r_inc) and the optical relay (OR), wherein:
   the deflected light ray (r_def) forms a deflection angle (alpha_def) with the normal (n_OR) in a deflection plane (Pl_def) formed by the deflected light ray (r_def) and the normal (n_OR);
      wherein:
      the optical relay (OR) comprises a diffraction grating and is positioned to have either
the incidence angle (alpha_inc) different from the deflection angle (alpha_def) and the incidence plane (Pl_inc) different from the deflection plane (Pl_def); or
the incidence angle (alpha_inc) equal to the deflection angle (alpha_def) and the incidence plane (Pl_inc) different from the deflection plane (Pl_def); or
the incidence angle (alpha_inc) different from the deflection angle (alpha_def) and the incidence plane (Pl_inc) coincides with the deflection plane (Pl_def).

By eye, one means an eyeball and its components, which comprise a pupil, an iris, a sclera, a cornea, and a retina. With the term eye, one may also include a region surrounding an eyeball, which comprises an upper eyelid, a lower eyelid, one or a plurality of skin patches, one or a plurality of skin folds, and one or two eyebrows. By eye, one also means any combination of an eyeball, the elements thereof, and the region surrounding the eyeball.

By light source (LSo), one means one or more individual sources of light or, more generally, of electromagnetic radiation. The light provided by each individual source may have specific time, frequency (and thus wavelength), polarization, and coherence characteristics.

Concerning the time characteristics, the light may have a modulation, such as amplitude modulation, phase modulation, frequency modulation, pulse-width modulation, and modulation by orthogonal codes.

Concerning the frequency characteristics and its corresponding wavelength characteristics, the light may have a spectrum that consists either of one or more frequencies, or of frequency bands at one or more frequencies, with frequency bandwidths that may be relatively narrow. Preferably, the wavelength is chosen in the infrared (IR) part of the electromagnetic spectrum, just outside the visible part. More preferably, the wavelength of the light source is in the range from 800 nm to 3000 nm. Most preferably, the wavelength of the light source is in the range from 850 nm to 890 nm. Even more preferably the wavelength is 890 nm.

Concerning the polarization characteristics, the light may be unpolarized, partially polarized, or polarized. The polarization may be linear or circular. Preferably, the light source is unpolarized.

Concerning the coherence, the light may be incoherent, partially coherent, or coherent, in time and/or space. Preferably, the light is incoherent.

When using several sources of light, each source of light may have the same or different time, frequency, polarization, and coherence characteristics. They may emit at the same time or at different times. Preferably, each light source comprises a light emitting diode (LED).

The invention is described in terms of light rays or, simply, rays, such as an illuminating light ray (r_ill), an incident light ray (r_inc), and a deflected light ray (r_ref). But “ray” also means “beam”. It should be clear that the description in terms of rays is a significant simplification of the actual propagation of electromagnetic waves, governed by Maxwell's equations and by the corresponding equations of wave propagation. While the conceptual and practical notion of ray is used in geometrical optics, more precise approximations of Maxwell's equations are used, e.g., in Fourier optics and in physical optics. The use of rays has the advantage to permit a simple, concise description of the invention.

More precisely, by deflected light ray (r_def), one means a light ray that is deflected by an optical relay in a way that is not constrained by the basics laws of optics, as explained above. The terms “to deflect”, “deflected”, and the like comprise a meaning of changing a direction of the light ray when the light ray hits a surface.

By incidence angle (alpha_inc), one means the angle formed by the incident ray with the normal to a surface of the optical relay at the point of intersection of the incident ray with the surface of the optical relay.

By deflection angle (alpha_def), one means the angle formed by the deflected ray with the normal to a surface of the optical relay at the point of intersection of the deflected ray with the surface of the optical relay.

By light sensor (LSe), one means a device for sensing light, a device for collecting photons, a device for making images, an imaging sensor, an imaging device, or a camera, which all produce data related to an eye, comprising signals, images, images-like data, image sequences, and videos. When the light sensor is used to produce images or image-like data of an eye, the light sensor may comprise a sensing surface that has some depth and is typically planar, and optical elements configured to bring each deflected ray (r_def) to the appropriate location on the sensing surface. Each deflected ray may contribute to the intensity level at one or a plurality of image elements, called pixels. Particularly, the light sensor collects deflected rays (r_def) or images from the eyelids, pupil, iris, and the like of the eye.

By optical relay (OR), one means a device made of a surface or a volume or both having one or more of the following capabilities, which may be wavelength dependent: a deflection capability, a transmission capability, an absorption capability, a transparency capability, a reflection capability, a refraction capability, a diffraction capability, a focusing capability, an imaging capability, and a selectivity capability. These capabilities are convenient ways of referring to specific transformations of one or a plurality of incident light rays governed by Maxwell's equations. Some of the above capabilities are related to each other. Preferably, the optical relay (OR) according to the invention has a wavelength-dependent diffraction capability.

By deflection capability, one means the capability of the optical relay to change the light ray direction in a way that does not obey the basic laws of reflection optics with respect to the surface of the optical relay.

By transmission capability, one means the capability of the optical relay to allow the incident light to traverse its surface, generally with some attenuation.

By absorption capability, one means the capability of the optical relay to absorb some of the incident light. Preferably, the absorption capability should not exceed 20% at visible wavelengths.

By transparency capability, one means the capability of the optical relay to allow most of the incident light to traverse the optical relay at a specific wavelength. By transparent, one also means negligible absorption and diffraction. Preferably, the optical relay is transparent at visible wavelengths. Preferably, the optical relay is transparent in the range from 380 and 780 nm.

By reflection capability, one means the capability of the optical relay to change the direction of the incident light according to the basic laws of reflection optics.

By refraction capability, one means the capability of the optical relay to change the direction of the incident light at one or a plurality of interfaces between adjacent materials having different indices of refraction according to the laws of refraction of optics.

By diffraction capability, one means the capability of the optical relay to transform the incident light according to the laws of diffraction of optics. The diffraction capability is generally achieved by including specific physical structures on, or in, the optical relay. Such structures are generally called diffraction gratings, grating patterns, or Bragg arrays. Such structures are generally used to implement holographic elements. The diffraction capability may enable other capabilities such as the deflection capability, focusing capability, imaging capability, and selection capability.

By focusing capability, one means the capability of the optical relay to make a set of parallel, or nearly parallel, incident light rays to focus at a common point of the light sensor (LSe).

By imaging capability, one means the capability of the optical relay to form an image formed by one or more pixels at the output of the light sensor (LSe) from a set of incident light rays.

By selectivity capability, one means the capability of the optical relay to perform some operations, such as deflection, at a specific frequency or band of frequencies. Preferably, the selectivity capability of the optical relay refers to deflection, focusing, and/or imaging in the IR part of the electromagnetic spectrum, as well as to its simultaneous transparency in the visible.

More practically, the optical relay may be a molded insert.

The optical relay may also be applied to one surface of a lens in such a way that it is removable.

Alternatively, the optical relay may be imprinted on, or near, a surface of a lens or in the volume of a lens.

By “lens” one means a piece of material, such as glass or plastic, positioned in the field of vision of the user, such as in eyeglasses or equivalent. This lens may simply transmit the incident light, or it may act upon it to achieve a specific optical property, such as focusing.

The optical relay may comprise one or several optical coatings.

The optical relay may consist of several pieces, each acting as an optical relay.

Generally, the optical relay is a holographic element or, synonymously, a diffractive element.

The holographic element may be a surface hologram or a volume hologram. The holographic element may be implemented using silver-halide material and techniques, or dichromated-gelatin (DCG) material and techniques, as described by T. G. Georgekutty and H. Liu, in Appl. Opt. 26, 372-376 (1987). Holographic elements can be created on the surface of the optical relay (surface hologram) or within the volume (generally near the surface) of the optical relay (volume hologram). The holographic effect is obtained by creating diffractions patterns or arrays on the surface or in the volume, as applicable. These arrays are often called Bragg arrays. Each array has a particular spatial periodicity (and, thus, a corresponding spatial period and a corresponding spatial frequency), which makes it to exhibit its special behavior, such as focusing, at a specific temporal frequency corresponding to this spatial period. The period of the array must therefore be adapted to the frequency (and, thus, wavelength) of interest, such as a particular IR frequency. Holographic elements exhibit their special behavior in a fairly narrow range of frequencies. By using a small number of distinct spatial periods, one can make the holographic element to exhibit its special behavior at the same number of distinct frequencies or frequency bands.

In the case of a volume hologram, which is our preferred way of implementing holographic elements in the context of the present invention, one often talks about Bragg planes or surfaces.

Generally, the Bragg arrays or planes are characterized by a spatial period, d and at least two refractive indices. The diffraction law is governed by a Bragg equation, limited to the first order of diffraction:

2d cos(ε₁)=λ₀/n

wherein:
n means a mean refractive index of a holographic element,
ε₁ means an angle of incidence measured between an incident ray and the normal to the Bragg planes (n_BP), normal that is perpendicular to the grating direction (16), and
λ₀ means the wavelength (in vacuum) of the incident ray and thus of the light source.

With this definition, inside the holographic element of index n, the angle of deflection (ε₂) as measured between the deflected ray and the normal to the Bragg planes (n_BP) is always equal to the angle of incidence ε₁.

The fact that a holographic element exhibits its special behavior at (and near) a specific frequency is particularly useful. For example, this allows the holographic element to work as a deflector or imager at specific frequencies, such as at one or a plurality of IR frequencies, and to be transparent at all other frequencies, in particular in the visible. This is exactly what is relied upon when illuminating the eye in the IR and recording the corresponding IR images of the eye via a holographic element placed in the field of vision of the user.

Particularly, an holographic element can perform deflection and focusing capabilities on the IR light coming from an eye as a result of it being illuminating in the IR by the light source, while being simultaneously transparent or quasi transparent in the visible, which allows a user to see through the holographic element placed in his field of vision, and the holographic element to be quasi invisible to the user.

One advantage of the present invention is that the system is not limited by both optical constraints of the state of the art. The system allows a greater freedom of configuration between the light source, the optical relay, and the light sensor.

The light source, the optical relay, and the light sensor according to the invention are configured to perform an observation of one eye; but the system may be duplicated to allow for the observation of each of both eyes. The system of an observation of an eye according to the invention may also be extended to the observation of other parts of the user such as head, parts thereof, or other body elements.

In a preferred embodiment, the light source, the optical relay, and the light sensor of the system for an observation of an eye according to the invention are connected to a support. The support may be fixed or mobile.

The present invention also concerns a device for an observation of an eye (E) comprising:
a frame (FR) configured to be worn on a user;
a light source (LSo) connected to the frame (FR) and positioned with respect to the eye (E) to emit an illuminating light ray (r_ill) towards the eye (E);
an optical relay (OR) connected to the frame (FR) and positioned with respect to the eye (E) to receive an incident light ray (r_inc) resulting from the interaction between the illuminating light ray (r_ill) and the eye (E), wherein:
   the incident light ray (r_inc) intersects the optical relay (OR) at an intersection point (P_int), and
   forms an incidence angle (alpha_inc) with the normal (n_OR) to the optical relay at an intersection point (P_int) in an incidence plane (Pl_inc) formed by the incident light ray (r_inc) and the normal (n_OR);
a light sensor (LSe) connected to the frame (FR) and positioned with respect to the optical relay (OR) to receive a deflected light ray (r_def) resulting from the interaction between the incident light ray (r_inc) and the optical relay (OR), wherein:
   the deflected light ray (r_def) forms a deflection angle (alpha_def) with the normal (n_OR) in a deflection plane (Pl_def) formed by the deflected light ray (r_def) and the normal (n_OR);
      wherein:
      the optical relay (OR) comprises a diffraction grating and is positioned to have either
the incidence angle (alpha_inc) different from the deflection angle (alpha_def) and the incidence plane (Pl_inc) different from the deflection plane (Pl_def); or
the incidence angle (alpha_inc) equal to the deflection angle (alpha_def) and the incidence plane (Pl_inc) different from the deflection plane (Pl_def); or
the incidence angle (alpha_inc) different from the deflection angle (alpha_def) and the incidence plane (Pl_inc) coincides with the deflection plane (Pl_def).

In a preferred embodiment, the light source, the optical relay, and the light sensor of the device for an observation of an eye according to the invention are connected to a frame to be worn by a user on his head.

More particularly, the device can be configured in such a way that the frame and the elements connected to it can be used over, or in conjunction with, conventional prescription glasses or sunglasses.

The frame of the device according to the invention may comprise a front piece and at least one sidepiece such as in the frame of glasses, spectacles, eye glasses, goggles, or equivalent. The front piece may comprise a support and one or two lenses or glasses. Preferably, these lenses or glasses are made of glass material or plastic material.

On such a frame, the light source is preferably arranged on the front piece of the frame and directed towards an eye of the user. The light source is most preferably arranged at the bottom of the front piece of the frame, and, particularly, out of the field of vision of the user.

On such a frame, the optical relay is preferably arranged on the front piece of the frame and most preferably integrated in the lens located in front of the observed eye if the transparency capability of the optical relay is high enough.

The optical relay may be positioned for example at one of several positions on the lens. This position may be at the center, top, bottom, left, or right of the lens.

On such a frame, the light sensor is preferably positioned on the sidepiece of the frame close to the observed eye in such a way as to receive as much as possible of the light coming from the eye as a result of its illumination by the light source. Since the light source, optical relay, and light sensor are preferably configured to operate at a specific frequency, preferably in the IR, and to some degree in a narrow frequency band around the specific frequency, the light sensor will record essentially the deflected light at the specific frequency resulting from the interaction between the illuminating light ray and the eye.

A filter passing and/or blocking some bands of wavelengths in the electromagnetic spectrum may be added in front of the light sensor (LSe). Preferably, this filter can be used to block light in the visible part of the electromagnetic spectrum.

The system and device according to the invention may also comprise processing means for converting an output from the light sensor into analog or digital data information about the state of the eye of the user.

It may further be used to obtain data about the state of the body or mind of the user.

The system and device may further comprise an ambient-light sensor producing an output signal that can be used to control one or more of the individual sources of light and the output of the light sensor.

The system and device may comprise an ambient-light sensor producing an output signal that can be used by the processing means, for example to control one or more of the light sources, the output of the light sensor, and the operation of the processing means. The ambient-light sensor may also be the light sensor (LSe) itself.

The device according to the invention may further comprise an attitude sensor or a plurality of attitude sensors that provide the position and orientation of the frame with respect to the physical world.

The device may also comprise another sensor that provides information about the environment, such as a camera providing images of the environment in front of the user.

Finally, the present invention also refers to all applications using the system or device for an observation of an eye such as systems or devices for monitoring drowsiness, alertness, fatigue, somnolence, alertness, wakefulness, distraction, inattention, or equivalent of the user.

The system or device may also be used in applications using eye-tracking and/or gaze-tracking, or the knowledge of which direction the user is looking into with respect to the frame or with respect to the physical environment or both, such as in psychological studies, in market research, or for the remote selection of items.

The system or device may further be used in applications requiring images of the interior of the eye, for example of the retina, such as in ophthalmology.

The invention will now be illustrated in detail with reference to the drawings.

The following description is presented to enable one person skilled in the art to make and use the invention. Descriptions of specific embodiments and applications are provided only as examples. But the present invention is not limited to such embodiments and applications. Other embodiments and applications are possible without departing from the scope of the invention.

A very accurate numerical simulation of the invention can be performed in a computer by using advanced ray-tracing software packages such as ASAP provided by Breach Research. The ASAP software package was used to illustrate the invention hereafter.

### BRIEF DESCRIPTION OF THE DRAWINGS

FIG. 1 shows a schematic drawing of the system according to the invention.

FIG. 2, FIG. 3, and FIG. 4 show schematic drawings of the optical relay for three different configurations according to the invention.

FIG. 5 and FIG. 6 show schematic drawings illustrating the focusing capability of an optical relay (OR) according to the invention.

FIG. 7 shows a schematic drawing illustrating the imaging capability of an optical relay (OR) according to the invention.

FIG. 8 shows a schematic drawing of one device according to the invention.

FIG. 9 shows a perspective view (FIG. 9a) and a projected view (FIG. 9b) of a practical implementation of the system according to the invention.

FIG. 10 shows the diffractive element (12) used in the practical implementation illustrated in FIG. 9.

FIG. 11 shows the diffraction efficiency (i.e. selectivity) as a function of wavelength for the diffractive element illustrated in FIG. 10.

FIG. 12 shows the diffraction efficiency (i.e. selectivity) as a function of the angle of incidence for the diffractive element illustrated in FIG. 10.

FIG. 13 shows the transmittance (i.e transmission) as a function of the wavelength for the diffractive element illustrated in FIG. 10.

FIG. 14 shows perspective views (FIG. 14 a, b) and projected views (FIG. 14 c, d) of an example of a first embodiment of the device according to the invention wherein the incidence angle is different from the deflection angle and the incidence plane is different from the deflection plane.

FIG. 15 shows perspective views (FIG. 15 a, b) and projected views (FIG. 15 c, d) of an example of a second embodiment of the device according to the invention wherein the incidence angle is equal to the deflection angle and the incidence plane is different from the deflection plane.

FIG. 16 shows perspective views (FIG. 16 a, b) and projected views (FIG. 16 c, d) of a third embodiment of the device according to the invention wherein the incidence angle is different from the deflection angle and the incidence plane coincides with the deflection plane.

In the description of the drawings, the following elements are used with their related definition:
a light source (LSo), comprising at least one individual source of light, preferably in the infrared (IR) part of the electromagnetic spectrum,
an eye (E),
an optical relay (OR) with at least the basic capability of deflecting an incident ray (r_inc) into a specified direction, by exploiting either its surface properties, or its volume properties, or both,
a deflecting surface (DS), which is typically, but without limitation, a reference plane corresponding to, and/or parallel to, an outside surface of an optical relay (OR),
a light sensor (LSe),
an illuminating ray (r_ill),
an incident ray (r_inc), which is a ray incident on an optical relay (OR) and which corresponds to an illuminating ray (r_ill),
a deflected ray (r_def), which is a ray deflected by an optical relay (OR) and which corresponds to an incident ray (r_inc),
an intersection point (P_int), defined as the intersection of an incident ray (r_inc) and an optical relay (OR) and/or a deflecting surface (DS),
a normal (i.e. a perpendicular) (n_OR), defined as an oriented line perpendicular to an optical relay (OR) and/or a deflecting surface (DS) at the intersection point (P_int),
an incidence plane (Pl_inc), defined by a normal (n_OR) and an incident ray (r_inc),
a deflection plane (Pl_def), defined by a normal (n_OR) and a deflected ray (r_def),
an incidence angle (alpha_inc), defined as the customary angle between a normal (n_OR) and an incident ray (r_inc),
a deflection angle (alpha_def), defined as the customary angle between a normal (n_OR) and a deflected ray (r_def),
an incidence trace (Tr_inc), defined as the line of intersection of an incidence plane (Pl_inc) and a deflecting surface (DS),
a deflection trace (Tr_def), defined as the line of intersection of a deflection plane (Pl_def) and a deflecting surface (DS),
a reference x-axis (x),
a reference y-axis (y), orthogonal to the x-axis (x) and forming a right-handed system with the x-axis (x) and a normal (n_OR),
an angle (phi_inc), defined as the customary angle between an incidence trace (Tr_inc) and the x-axis (x),
an angle (phi_def), defined as the customary angle between a deflection trace (Tr_def) and the x-axis (x),
a difference angle (Delta_phi), defined as the difference between an angle (phi_def) and an angle (phi_inc),
a bundle of rays, or ray bundle (RB), defined as being a set of rays all oriented in a same direction,
a focal point (FP), defined as the point through which all the rays in a ray bundle (RB) incident on an optical relay (OR) go through after deflection by the optical relay (OR) in the case where the optical relay (OR) provides an imaging capability or equivalent,
a frame (FR), i.e. a support,
a lens (LE), as found, e.g., in a pair of eye glasses.

### DETAILED DESCRIPTION OF THE DRAWINGS

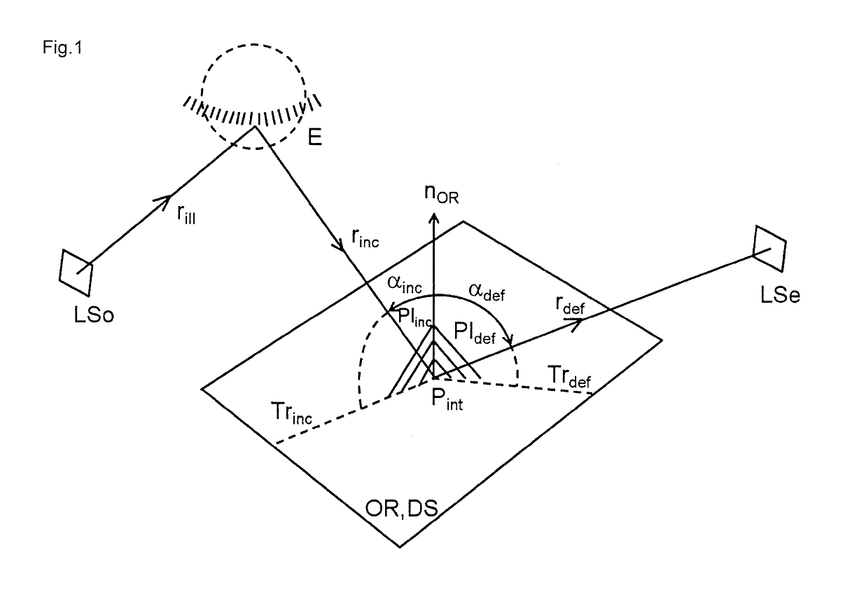

FIG. 1 shows a schematic drawing of a system according to the invention. The figure shows a light source (LSo), an eye (E), an optical relay (OR), and a light sensor (LSe). The figure also shows a deflecting surface (DS), which is typically a reference plane corresponding to, and/or parallel to, an outside surface of the optical relay (OR). An illuminating ray (r_ill) is produced by the light source (LSo) and further illuminates an eye (E). After interaction with the eye (E), this ray gives rise to an incident ray (r_inc) that intersects the deflecting surface (DS) at an intersection point (P_int). After interaction with the optical relay (OR), this ray gives rise to a deflected ray (r_def) that reaches the light sensor (LSe). The normal (n_OR) to the deflecting surface (DS) at the intersection point (P_int) and the incident ray (r_inc) define an incidence angle (alpha_inc) and an incident plane (Pl_inc). The same normal and the deflected ray (r_def) define a deflection angle (alpha_def) and a deflection plane (Pl_def). According to the invention, the incidence angle (alpha_inc) differs from the deflection angle (alpha_def) and/or the incidence plane (Pl_inc) differs from the deflection plane (Pl_def). Since the incidence plane (Pl_inc) contains the normal (n_OR) to the deflecting surface (DS), it is perpendicular to the deflecting surface (DS). The same is true for the deflection plane (Pl_def). The intersection of the incidence plane (Pl_inc) and the deflecting surface (DS) is referred to as the incidence trace (Tr_inc). The intersection of the deflection plane (Pl_def) and the deflecting surface (DS) is referred to as the deflection trace (Tr_def).

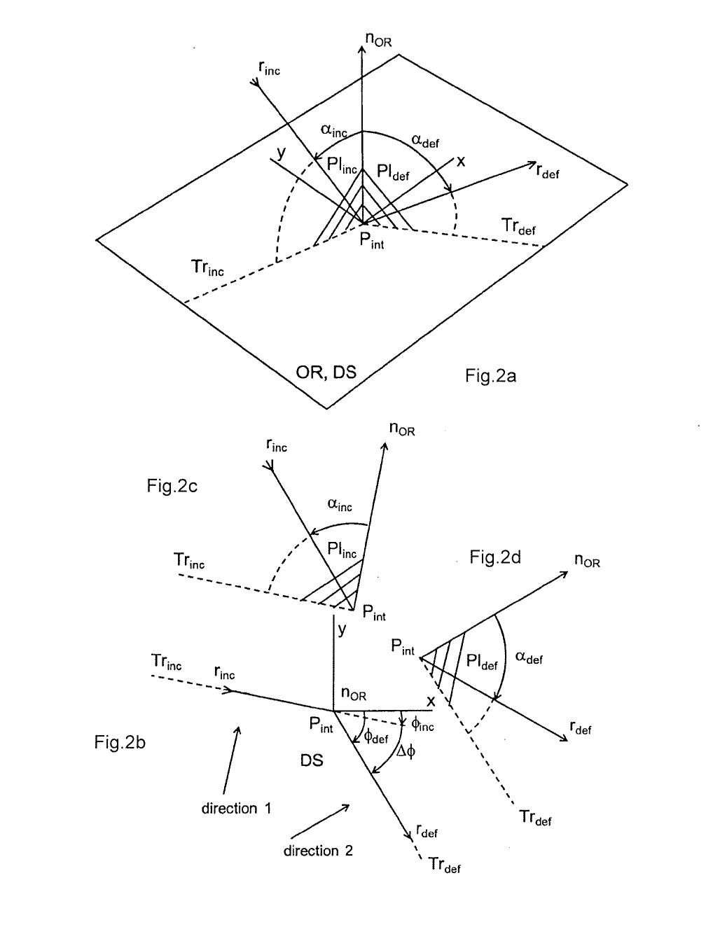

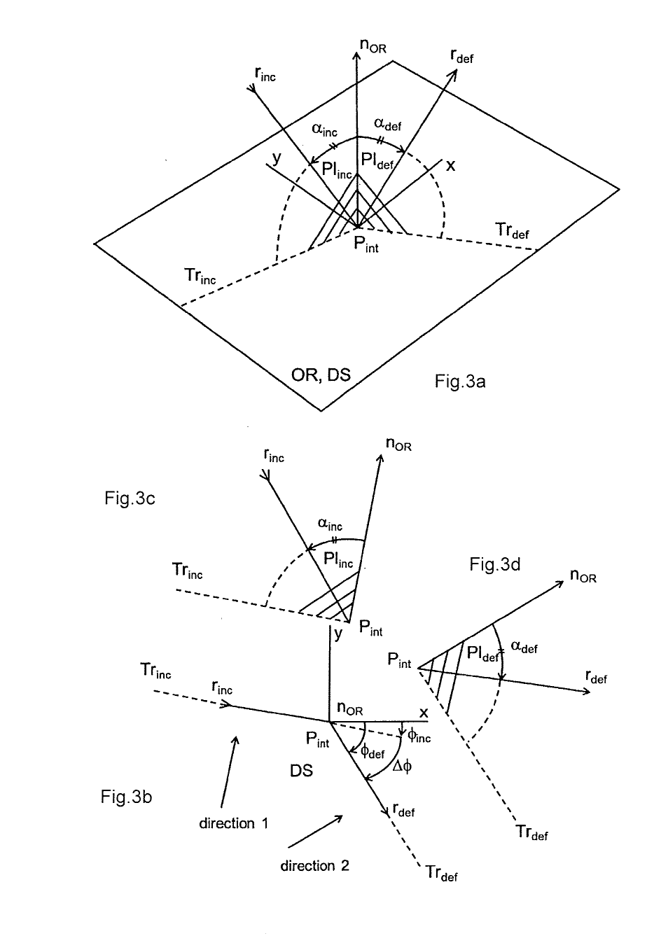

FIG. 2(a) shows a first embodiment of the system according to the invention wherein the incidence plane (Pl_inc) differs from the deflection plane (Pl_def) and the incidence angle (alpha_inc) differs from the deflection angle (alpha_def). In such an embodiment, the difference angle (Delta_alpha) between these last two angles, i.e. Delta_alpha=alpha_def−alpha_inc, differs from zero, whether this angle is expressed in degrees, radians, or other units. Reference axes are introduced in FIG. 2(a) for defining the orientation of planes perpendicular to the deflecting surface (DS). These axes are referred to as x-axis (x) and y-axis (y), and are orthogonal and form a right-handed system with the normal (n_OR).

FIG. 2(b) shows a top view—also called a plan view—of FIG. 2(a), highlighting two specific viewing directions. Viewing direction 1 is perpendicular to Tr_inc, and viewing direction 2 is perpendicular to Tr_def.

FIGS. 2(c) and 2(d) show elevation views—also called front views—corresponding to direction 1 and direction 2, respectively.

FIG. 2(b) shows an angle (phi_inc) between the x-axis (x) and the incidence trace (Tr_inc), an angle (phi_def) between the x-axis (x) and the deflection trace (Tr_inc), and a difference angle (Delta_phi) between these last two angles, i.e. Delta_phi=phi_def−phi_inc. These angles are signed quantities defined in a customary way. The difference angle (Delta_phi) is thus the angle between the incidence plane (Pl_inc) and the deflected plane (Pl_def).

As FIG. 2 corresponds to the case where the incidence plane (Pl_inc) differs from the deflected plane (Pl_def), the difference angle (Delta_phi) differs from zero, whether this angle is expressed in degrees, radians, or some other units.

FIG. 2(c) shows the elevation view corresponding to viewing direction 1, which is perpendicular to Tr_inc. This figure also shows the incidence angle (alpha_inc).

FIG. 2(d) shows the elevation view corresponding to viewing direction 2, which is perpendicular to Tr_def. This figure also shows the deflection angle (alpha_def).

FIG. 3(a) shows a second embodiment of the system according to the invention wherein the incidence plane (Pl_inc) differs from the deflection plane (Pl_def) and the incidence angle (alpha_inc) is equal to the deflection angle (alpha_def). In such embodiment, the difference angle (Delta_phi) differs from zero and the difference angle (Delta_alpha) is zero.

FIG. 3(b) shows a top view—also called a plan view—of FIG. 3(a), highlighting two specific viewing directions. Viewing direction 1 is perpendicular to Tr_inc, and viewing direction 2 is perpendicular to Tr_def. FIGS. 3(c) and 3(d) show elevation views—also called front views—corresponding to direction 1 and direction 2 as already defined in FIG. 2 but with the incidence angle (alpha_inc) equal to the deflection angle (alpha_def).

FIG. 4(a) shows a third embodiment of the system according to the invention wherein the incidence plane (Pl_inc) coincides with the deflection plane (Pl_def) and the incidence angle (alpha_inc) differs from the deflection angle (alpha_def). In such embodiment, the difference angle (Delta_phi) equals zero and the difference angle (Delta_alpha) differs from zero.

FIG. 4(b) shows a top view—also called a plan view—of FIG. 4(a), highlighting one viewing direction. Viewing direction 1 is perpendicular to Tr_inc and to Tr_def because the incidence plane (Pl_inc) and the deflection plane (Pl_def) coincide.

FIG. 4(c) shows an elevation view—also called front view—corresponding to direction 1 as already defined in FIG. 2 but with the incidence angle (alpha_inc) different from the deflection angle (alpha_def).

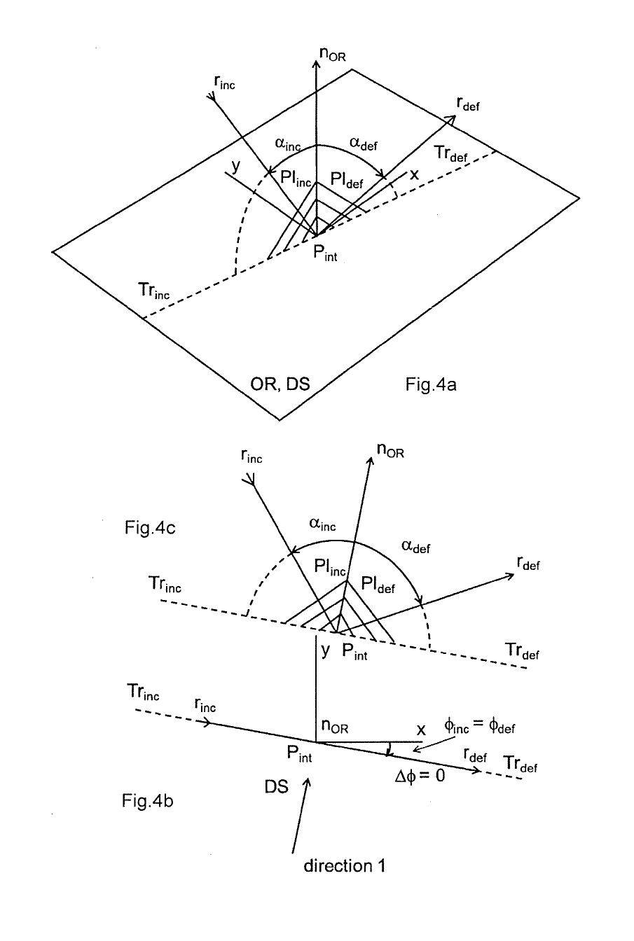

FIG. 5 illustrates the focusing capability of an optical relay (OR). The figure shows that each incident ray (r_inc) in a ray bundle (RB) incident on the optical relay (OR) is deflected in such a way that all the rays in the ray bundle (RB) go through a common point, called a focal point (FP).

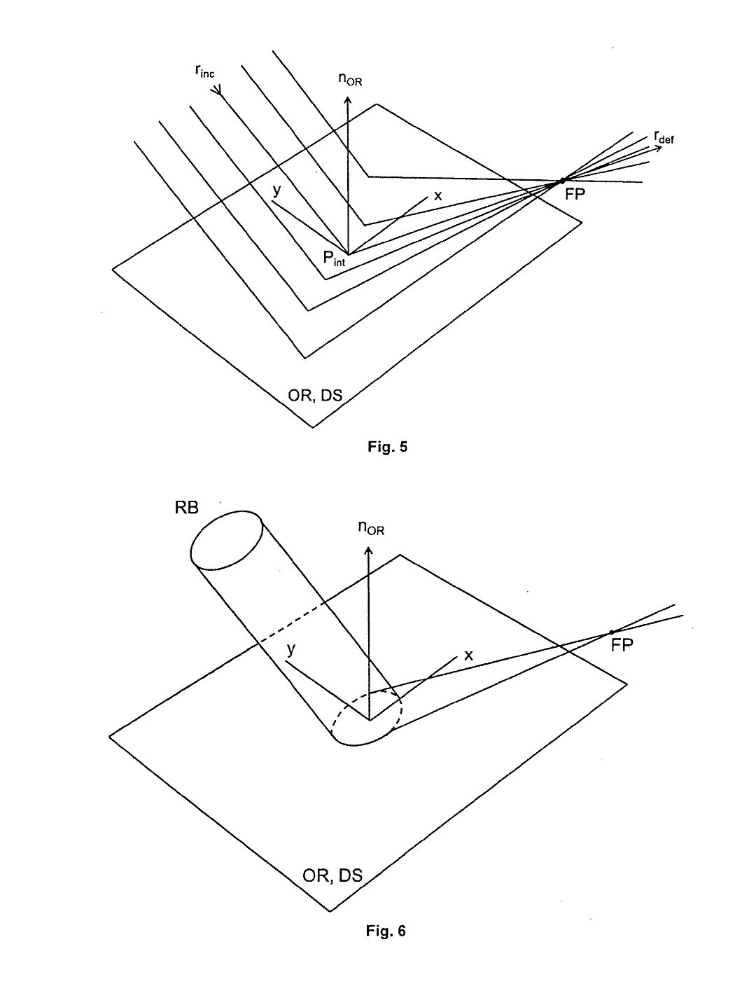

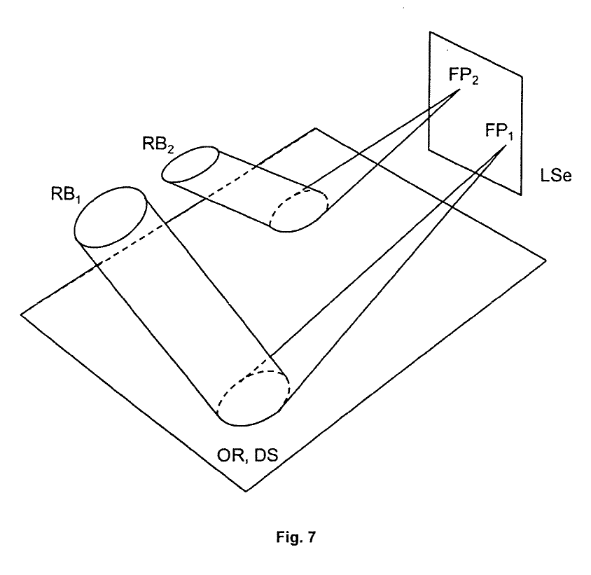

FIG. 6 also illustrates the focusing capability of an optical relay (OR), but in a more concise way than in FIG. 5. The parallel incident rays (r_inc) in the ray bundle (RB) are indeed represented as a cylinder, and the corresponding set of deflected rays as a cone. This concise representation is used advantageously in FIG. 7.

FIG. 7 illustrates the imaging capability of an optical relay (OR). The figure shows that the optical relay (OR) focuses ray bundles (here, RB_{—}1 and RB_{—}2) corresponding to different travel directions on generally different focal points (here, FP_{—}1 and FP_{—}2), all located on the light sensor (LSe).

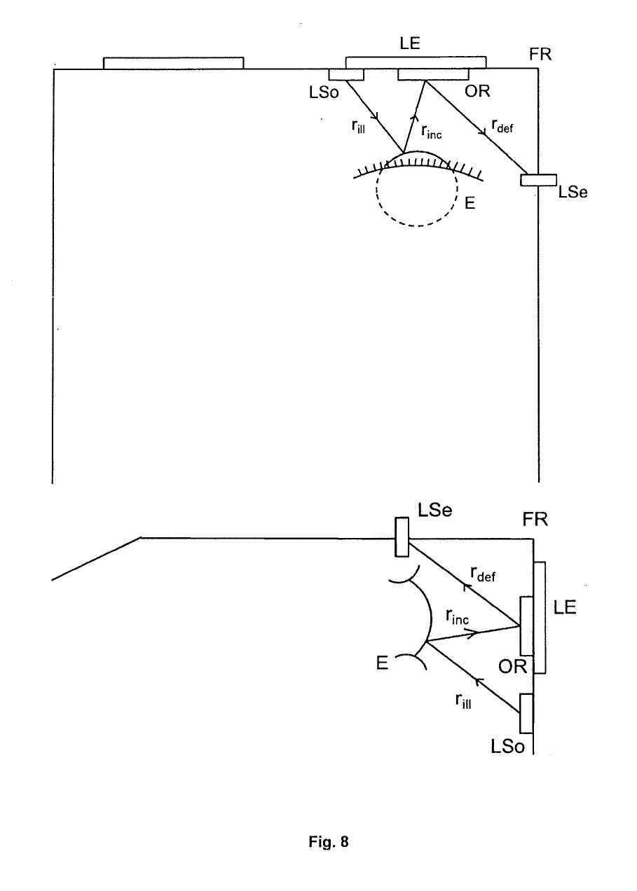

FIG. 8 shows a schematic drawing (top view and side view) of a device according to the invention. The figure shows a frame (FR) configured to be worn by a user and a light source (LSo) connected to the frame (FR) and positioned to emit an illuminating ray (r_ill) towards an eye (E).

An optical relay (OR) is also connected to the frame and is positioned with respect to the eye (E) to receive an incident ray (r_inc) resulting from the interaction of an illuminating ray (r_ill) with the eye (E). Finally, a light sensor (LSe) is also connected to the frame (FR) to receive a deflected light ray (r_def). All three elements are attached directly or indirectly to a frame (FR). If the frame is part of a pair of eyeglasses, the optical relay (OR) is preferably mounted on one of the lenses (LE) of the eyeglasses.

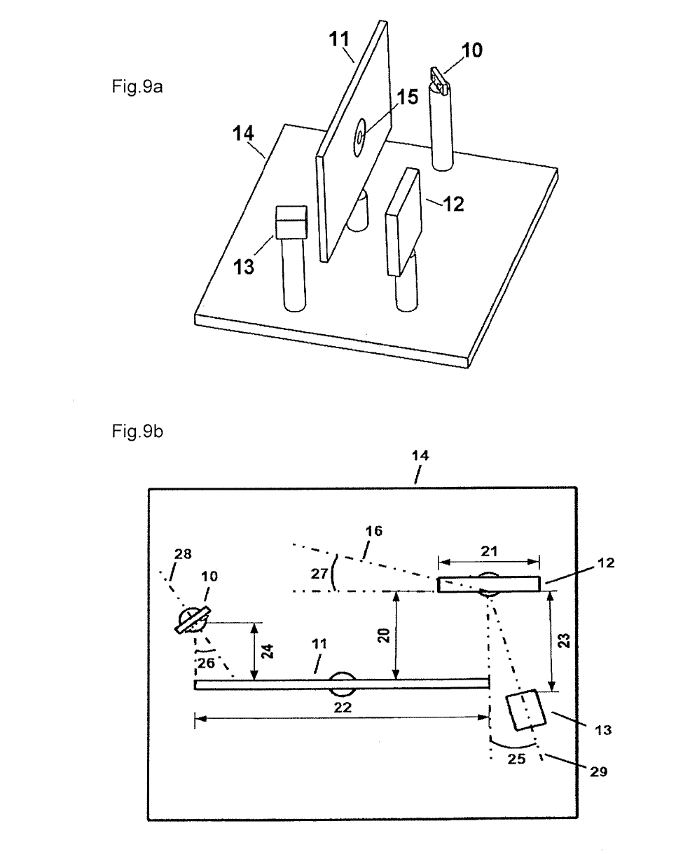

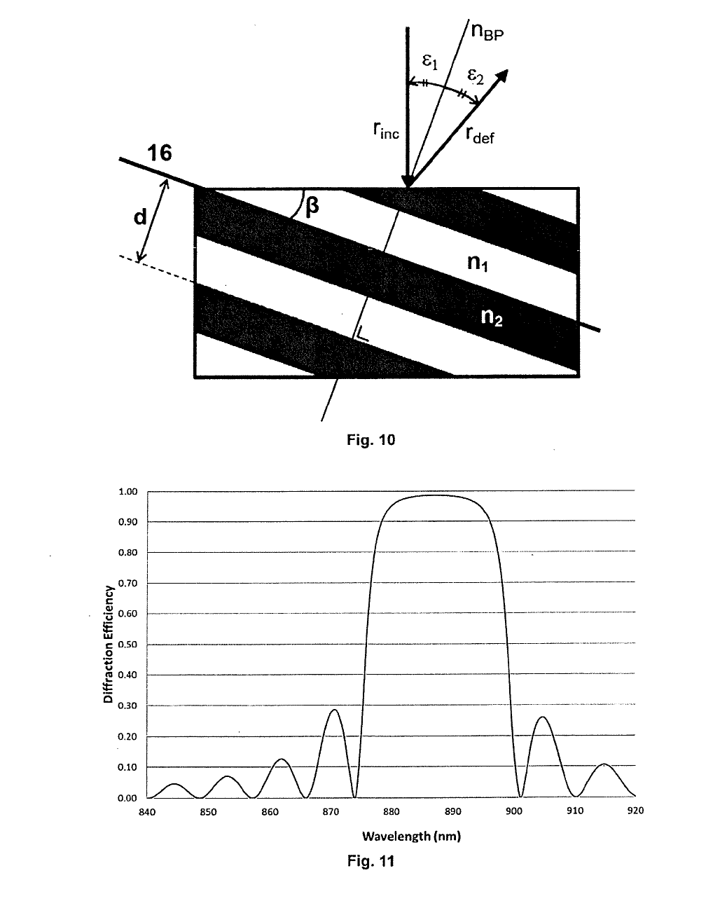

FIG. 9 (FIG. 9 a, b) shows a practical implementation, or experimental setup, of the system according to the invention. Two infrared (IR) emitters (10) provided by Vishay Semiconductors with a commercial reference LED VSMF3710 and having a peak wavelength at 890 nm with a spectral bandwidth of 40 nm are positioned on an optical table (14) to illuminate a picture of an eye (11) of dimensions 88 mm (22)×52 mm. A video camera (13) provided by Supercircuits with the commercial reference PC206XP and having an image sensor type B/W CMOS is also attached to the optical table (14), to record images of a diffractive element (12) of dimensions 30 mm (21)×30 mm. The camera is equipped with a filter provided by Präzisions Glas & Optik with commercial reference SCHOTT RG 830 and having a transmittance of 50% at 830 nm. The diffractive element (12) is a diffractive grating with an angle (β) of 20° (27) between the grating direction (16) and the deflecting surface (here the top surface of the diffractive element, or any surface parallel to this top surface) as shown in FIG. 10. Both infrared emitters (10) are placed 20 mm (24) from the eye (11), and with an angle of 40° (26) between the main radiation axis (28) of each infrared emitter (10) and the perpendicular to the (deflecting) surface of (11). The diffractive element (12) is placed 30 mm (20) from the eye (11) and 35 mm (23) from the camera (13). The angle (25) between the viewing axis (29) of the camera (13) and the perpendicular to the surface of the diffractive element (12) is 10°. The grating direction (16), the radiation axis (28), the viewing axis (29) of the camera (13), and the center (15) of the eye are in the same plane. This plane runs parallel to the surface of the optical table (14). FIG. 9(a) is a perspective view and FIG. 9(b) is a projected view.

FIG. 10 shows a cut through a diffractive element (12) used in the practical implementation of the invention shown in FIG. 9, including its diffraction gratings (or patterns), also called “Bragg planes”, inscribed in the volume of the diffractive element. The diffractive element illustrated here consists in a succession of bands with indices of refraction alternating between the symbolic values of n1 and n2, which are here 1.4 and 1.6 for dichromated gelatin. However, more complex periodic distributions of refraction index values can also be used. The spatial period d appearing in the Bragg equation corresponds to the total width of two successive bands, as shown in the figure. In this example, the angle β is 20° and the spatial period d is 315.7 nm. The figure illustrates the fact that the angle of incidence (zero in the case shown) is not equal to the angle of deflection, both being measured with respect to the normal to the diffractive element.

FIG. 11 shows a graph of the diffraction efficiency (on a scale from zero to one) as a function of the wavelength (in nm) for the diffractive element of FIG. 10, as simulated by a Rigorous Couple-Wave Analysis (RCWA) software. The graph shows the selectivity of this diffractive element as a function of the wavelength. The graph also shows that the diffractive element exhibits a maximum efficiency between 880 nm and 895 nm. The wavelength of the light source was consequently chosen within this band for the system to operate in the best possible way.

FIG. 12 shows a graph of the diffraction efficiency (on a scale from zero to one) as a function of the angle of incidence (in degrees) for the diffractive element of FIG. 10, as simulated by a Rigorous Couple-Wave Analysis (RCWA) software. The graph shows the selectivity of this diffractive element as a function of the angle of incidence. The graph also shows that the diffractive element exhibits a maximum of efficiency at angles of incidence of 0° and 40°, symmetrically with respect to the normal to the deflecting surface. Consequently, in the configuration of FIG. 9, the incidence angle is 0° and the deflection angle is 40°.

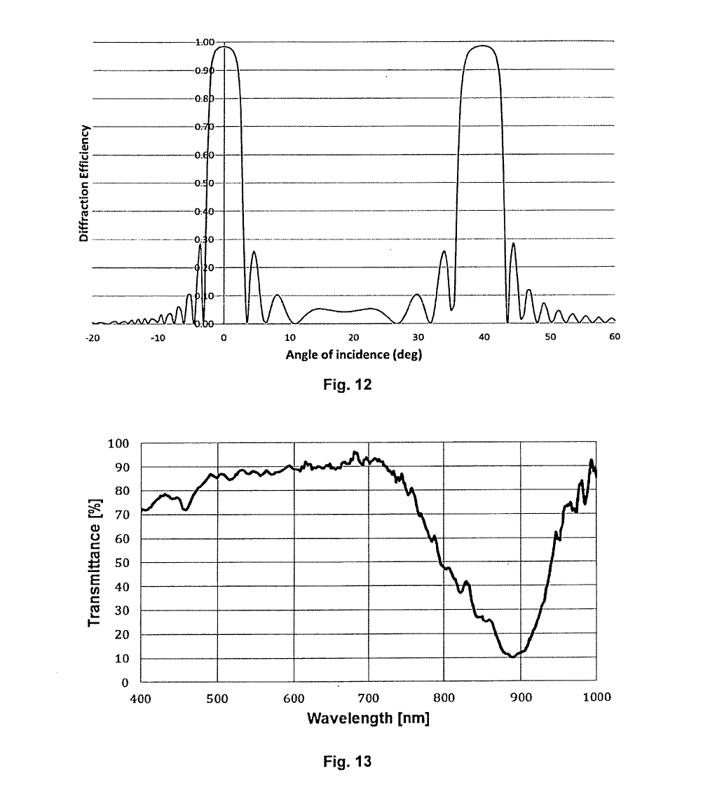

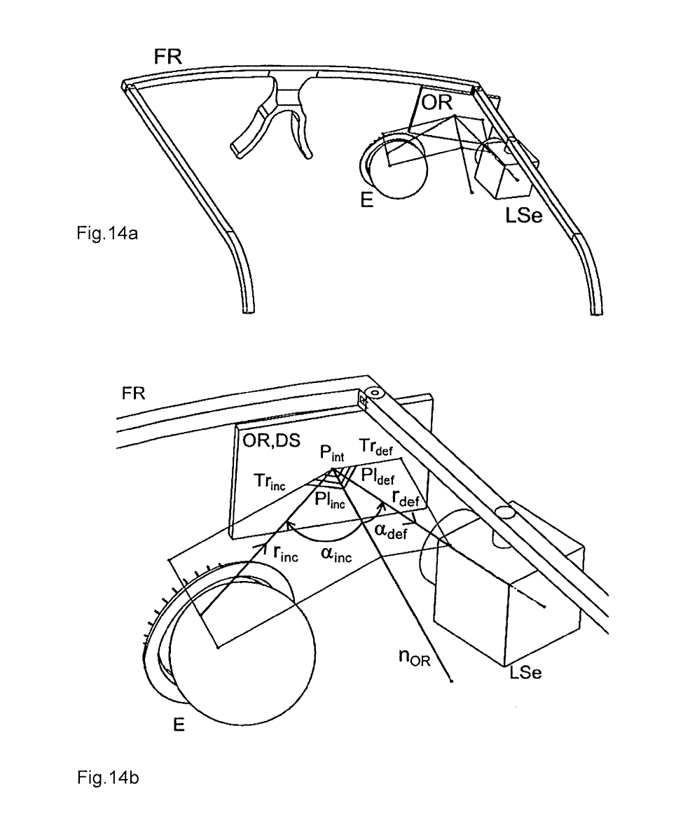

FIG. 13 shows a graph of the transmittance (in %) as a function of the wavelength (in nm) for a diffractive element with the structure of FIG. 10, prepared according to the literature, such as, for example, according to the paper by T. G. Georgekutty and H. Liu, in Appl. Opt. 26, 372-376 (1987). The transmittance is the ratio of the transmitted intensity to the incident intensity. It was measured with a spectrometer. The minimum of the transmittance is at 890 nm, which corresponds to the emission wavelength of a LED light source as for example the one provided by Vishay Semiconductors with a commercial reference VSMF3710. The light that is not transmitted is mainly diffracted towards the +1 diffraction order of the diffractive element. No other diffraction order is generated under this specific configuration.

The graph shows that the transmittance—and thus the transmission—goes up to close to 90%. The fact that one does not reach 100% is due to various losses. A diffractive element reaching a transmittance of 100% for some ranges of wavelengths would no longer transmit at the design wavelength with angular (Bragg) geometry. The region where the transmittance drops significantly has a full width at half maximum (FWHM) of about 140 nm, and this width is fully positioned within the IR part of the electromagnetic spectrum. Therefore, the diffractive element acts as a reflector, or mirror, in the near IR part of the electromagnetic spectrum.

In the visible part of the electromagnetic spectrum (from 380 nm to 780 nm), we expect the holographic grating to be efficiently transmitting the light, in other words to be transparent. As indicated above, the fact that the transmittance does not reach 100%, and even 90% in the present case, is due to various loss factors. The main loss factors are:
Fresnel reflections due to the change in refractive index at the interface between the diffractive element (gelatin and glass having a refractive index of 1.5) and the exterior (air having a refractive index of 1) and producing a loss (spurious reflection) of about 10%. If needed, the loss could be reduced to below 1% by using a specific anti-reflection coating.
Light scattering occurring inside the component, which produces a diffuse light reemission. The scattering level strongly depends upon the wavelength (λ); indeed according to the Rayleigh law, the scattering is proportional to 1/λ⁴. Consequently, the scattering is increasing in the blue range of the visible part of the electromagnetic spectrum. This fact is clearly depicted by the graph, with a transmission reduced at shorter wavelength. However, this reduction is tolerable.

Three practical examples are now described to illustrate three embodiments of the device according to the invention.

Three frames of spectacles to be worn by a user were developed using an optical relay (OR) implemented as a diffractive element such as the one described in FIGS. 10 to 13 but with a different angle (β), and attached to the front part of the spectacles. The video camera (LSe) from Supercircuits is positioned on one branch (or sidepiece) of the spectacles. An LED emitting IR light at 890 nm is used as light source but is not shown for clarity reason. The LED is connected to the bottom part (not shown) of the spectacles, in front of the eye.

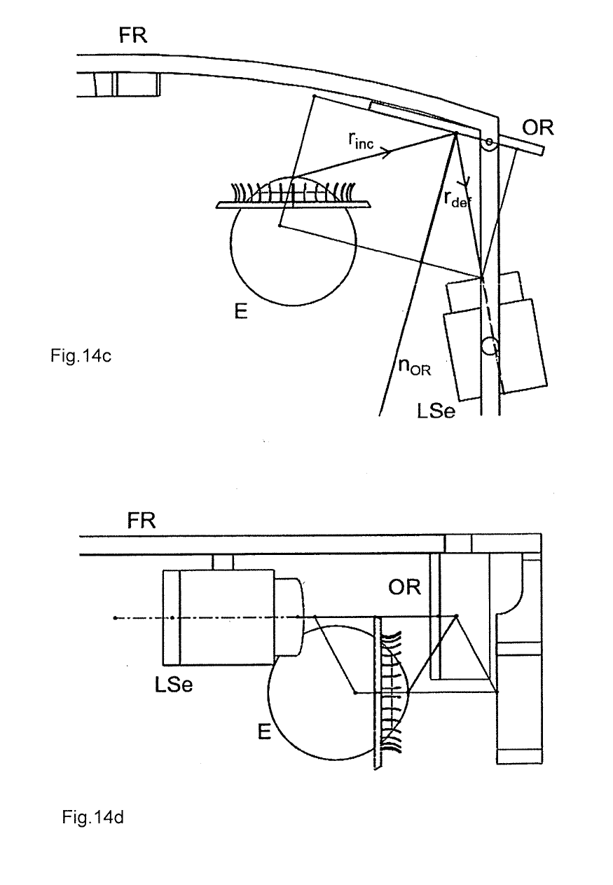

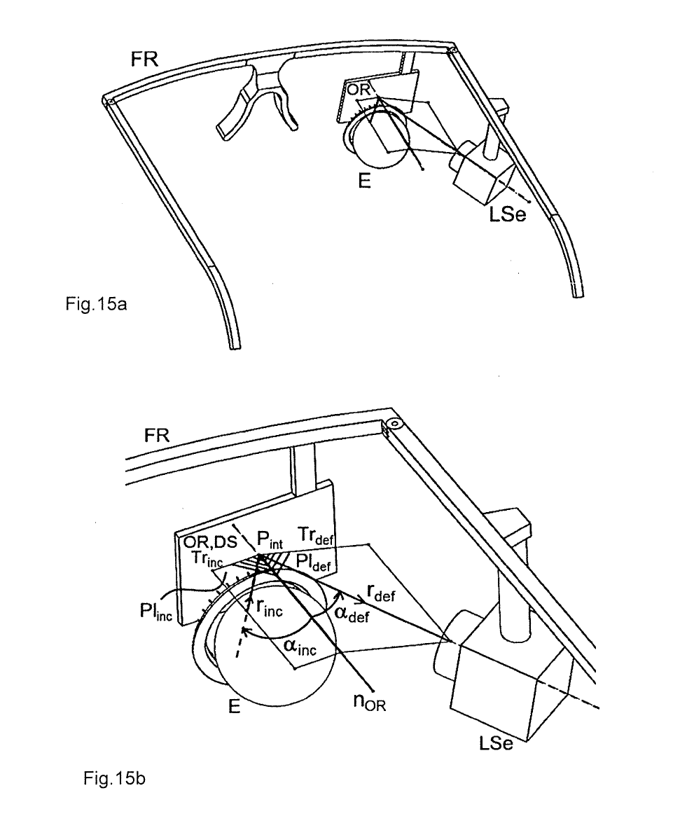

FIG. 14 shows perspective views (FIG. 14 a, b) and projected views (FIG. 14 c, d) of an example of a first embodiment of the device according to the invention. In this example of the first embodiment, the incidence angle is different from the deflection angle and the incidence plane is different from the deflection plane.

FIG. 14(a) is a perspective view of the entire device, while FIG. 14(b) is a close-up perspective view of part of the device. These views show, among others, the optical relay (OR)—implemented as a diffractive element—and its corresponding deflecting surface (DS) with its normal (n_OR). In this example, the diffractive element has an angle (β) of 21.5° between the grating direction and the deflecting surface. An incident ray (r_inc) coming from the eye (E), intersects the deflecting surface (DS) at an intersection point (P_int) and gives rise to a deflected ray (r_def) that reaches the light sensor (LSe)—implemented as a video camera—. The normal (n_OR) to the deflecting surface (DS) at the intersection point (P_int) and the incident ray (r_inc) define an incidence plane (Pl_inc) and an incidence angle (alpha_inc). The same normal and the deflected ray (r_def) define a deflection plane (Pl_def) and a deflection angle (alpha_def). The intersection of the incidence plane (Pl_inc) and the deflecting surface (DS) is referred to as the incidence trace (Tr_inc). The intersection of the deflection plane (Pl_def) and the deflection surface (DS) is referred to as the deflection trace (Tr_def). In this example of the first embodiment, the position of the diffractive element (OR) with respect to the eye (E) and the position of the camera (LSe) on one branch of the spectacles with respect to the diffractive element (OR) are adjusted to obtain an incidence angle (alpha_inc) that is different from the deflection angle (alpha_def) and an incidence plane (Pl_inc) that is different from the deflection plane (Pl_def). In the present example, the incidence angle (alpha_inc) is equal to 62.3° and the deflection angle (alpha_def) is equal to 25.0°. The angle between the incidence plane (Pl_inc) and the deflection plane (Pl_def) is equal to 153.9°.

FIGS. 14(c) and 14(d) are respectively a top view and a side view of the same example of the first embodiment, showing the positions of the different elements of the present device.

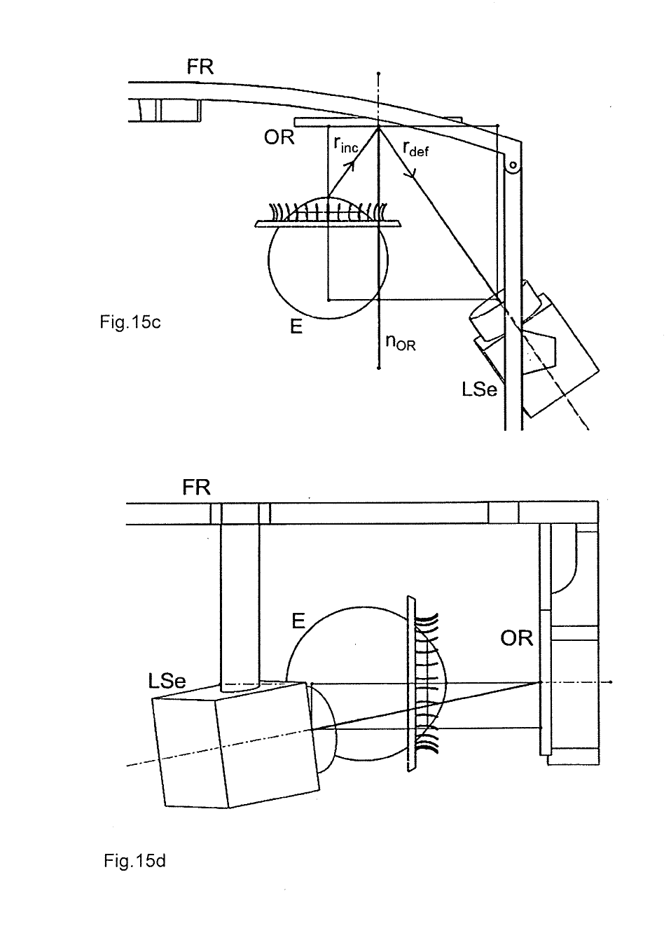

FIG. 15 shows perspective views (FIG. 15 a, b) and projected views (FIG. 15 c, d) of an example of a second embodiment of the device according to the invention. In this example of the second embodiment, the diffractive element has an angle (β) of 5.7° between the grating direction and the deflecting surface. The incidence angle is equal to the deflection angle and the incidence plane is different from the deflection plane.

FIG. 15(a) is a perspective view of the entire device, while FIG. 15(b) is a close-up perspective view of part of the device. The position of the diffractive element or optical relay (OR) with respect to the eye (E) and the position of the camera or light sensor (LSe) with respect to the optical relay (OR) lead to an incidence angle (alpha_inc) that is equal to the deflection angle (alpha_def) and to an incidence plane (Pl_inc) that is different from the deflection plane (Pl_def). In the present example, the incidence angle (alpha_inc) and the deflection angle (alpha_def) are both equal to 35.0°. The angle between the incidence plane (Pl_inc) and the deflection plane (Pl_def) is equal to 163.7°.

FIGS. 15(c) and 15(d) are respectively a top view and a side view of the same example of the second embodiment, showing the positions of the different elements of the present device.

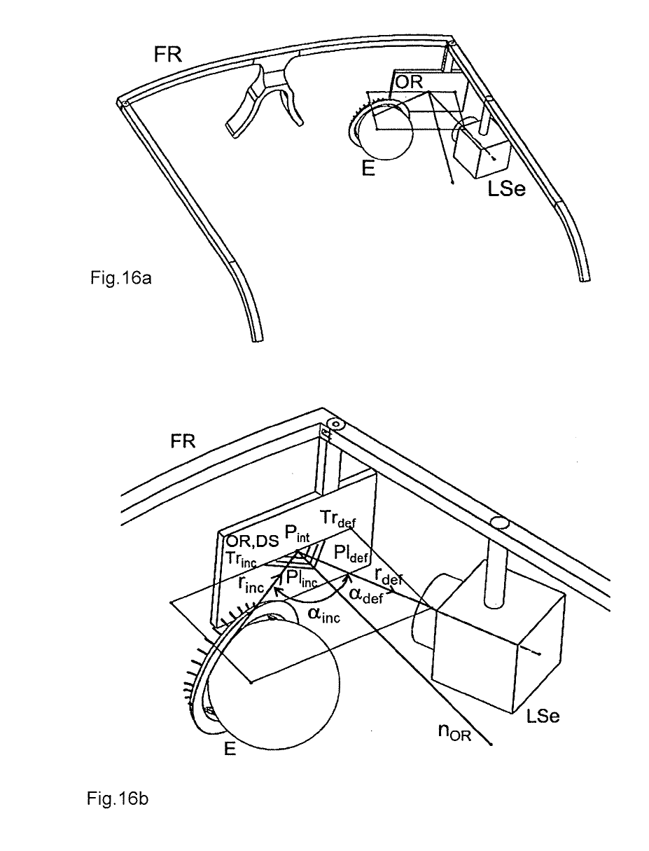

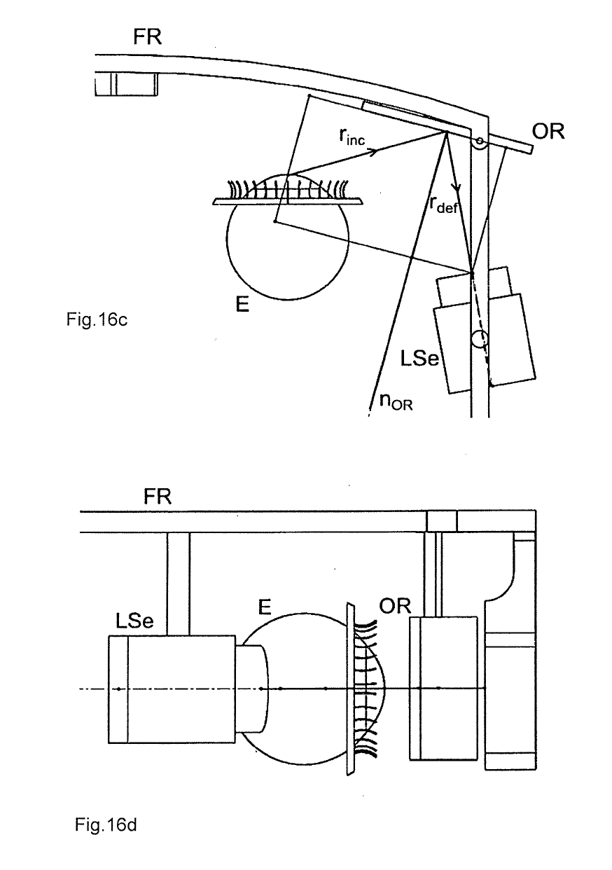

FIG. 16 shows perspective views (FIG. 16 a, b) and projected views (FIG. 16 c, d) of an example of a third embodiment of the device according to the invention. In this example of the third embodiment, the diffractive element has an angle (β) of 17.4° between the grating direction and the deflecting surface. The incidence angle is different from the deflection angle and the incidence plane coincides with the deflection plane.

FIG. 16(a) is a perspective view of the entire device, while FIG. 16(b) is a close-up perspective view of part of the device. The position of the diffractive element or optical relay (OR) with respect to the eye (E) and the position of the camera or light sensor (LSe) with respect to the optical relay (OR) lead to an incidence angle (alpha_inc) that is different from the deflection angle (alpha_def) and to an incidence plane (Pl_inc) that coincides with the deflection plane (Pl_def). In the present example, the incidence angle (alpha_inc) is equal to 59.7° and the deflection angle (alpha_def) is equal to 25.0°. The angle between the incidence plane (Pl_inc) and the deflection plane (Pl_def) is equal to 0°.

FIGS. 16(c) and 16(d) are respectively a top view and a side view of the same example of the third embodiment, showing the positions of the different elements of the present device.

1. A system for an observation of an eye (E) comprising:
a light source (LSo) positioned with respect to the eye (E) and configured to emit an illuminating light ray (r_ill) towards the eye (E);
an optical relay (OR) positioned with respect to the eye (E) and configured to receive an incident light ray (r_inc) resulting from the interaction between the illuminating light ray (r_ill) and the eye (E), wherein:
the incident light ray (r_inc) intersects the optical relay (OR) at an intersection point (P_int), and
forms an incidence angle (alpha_inc) with the normal (n_OR) to the optical relay (OR) at the point (P_int) in an incidence plane (Pl_inc) formed by the incident light ray (r_inc) and the normal (n_OR);
a light sensor (LSe) positioned with respect to the optical relay (OR) and configured to receive a deflected light ray (r_def) resulting from the interaction between the incident light ray (r_inc) and the optical relay (OR), wherein:
the deflected light ray (r_def) forms a deflection angle (alpha_def) with the normal (n_OR) in a deflection plane (Pl_def) formed by the deflected light ray (r_def) and the normal (n_OR);
characterized in that:
the optical relay (OR) comprises a diffraction grating and is positioned to have either
the incidence angle (alpha_inc) different from the deflection angle (alpha_def) and the incidence plane (Pl_inc) different from the deflection plane (Pl_def); or
the incidence angle (alpha_inc) equal to the deflection angle (alpha_def) and the incidence plane (Pl_inc) different from the deflection plane (Pl_def); or
the incidence angle (alpha_inc) different from the deflection angle (alpha_def) and the incidence plane (Pl_inc) coincides with the deflection plane (Pl_def).

2. The system according to claim 1 characterized in that the diffraction grating is a deflector at specific electromagnetic frequencies, preferably at one or a plurality of infrared (IR) frequencies, and is transparent at other electromagnetic frequencies.

3. The system according to claim 1 further comprising processing means for converting an output signal of the light sensor into analog or digital information data.

4. The system according to claim 1 characterized in that the diffraction grating is a Bragg array.

5. The system according to claim 4 characterized in that the Bragg array is a holographic element, preferably a volume holographic element.

6. The system according to claim 1 characterized in that the light source emits an illuminating light ray (r_ill) with a wavelength in the range from 800 nm to 3000 nm, preferably at 890 nm.

7. The system according to claim 1 further comprising a filter positioned before the light sensor (LSe) to block light in the visible part of the electromagnetic spectrum.

8. A device for an observation of an eye (E) comprising:
a frame (FR) configured to be worn on a user;
a light source (LSo) connected to the frame (FR) and positioned with respect to the eye (E) to emit an illuminating light ray (r_ill) towards the eye (E);
an optical relay (OR) connected to the frame (FR) and positioned with respect to the eye (E) to receive an incident light ray (r_inc) resulting from the interaction between the illuminating light ray (r_ill) and the eye (E), wherein:
the incident light ray (r_inc) intersects the optical relay (OR) at an intersection point (P_int), and
forms an incidence angle (alpha_inc) with the normal (n_OR) to the optical relay at the point (P_int) in an incidence plane (Pl_inc) formed by the incident light ray (r_inc) and the normal (n_OR);
a light sensor (LSe) connected to the frame (FR) and positioned with respect to the optical relay (OR) to receive a deflected light ray (r_def) resulting from the interaction between the incident light ray (r_inc) and the optical relay (OR), wherein:
the deflected light ray (r_def) forms a deflection angle (alpha_def) with the normal (n_OR) in a deflection plane (Pl_def) formed by the deflected light ray (r_def) and the normal (n_OR);
characterized in that:
the optical relay (OR) comprises a diffraction grating and is positioned to have either
the incidence angle (alpha_inc) different from the deflection angle (alpha_def) and the incidence plane (Pl_inc) different from the deflection plane (Pl_def); or
the incidence angle (alpha_inc) equal to the deflection angle (alpha_def) and the incidence plane (Pl_inc) different from the deflection plane (Pl_def); or
the incidence angle (alpha_inc) different from the deflection angle (alpha_def) and the incidence plane (Pl_inc) coincides with the deflection plane (Pl_def).

9. The device according to claim 8 characterized in that the diffraction grating is a deflector at specific electromagnetic frequencies, preferably at one or a plurality of infrared (IR) frequencies, and is transparent at other electromagnetic frequencies.

10. The device according to claim 8 further comprising processing means for converting an output signal of the light sensor into analog or digital information data.

11. The device according to claim 8 characterized in that the diffraction grating is a Bragg array, preferably a holographic element, more preferably a volume holographic element.

12. The device according to claim 8 wherein the light source emits an illuminating light ray (r_ill) with a wavelength in the range from 800 nm to 3000 nm, preferably at 890 nm.

13. The device according to claim 8 further comprising a filter positioned before the light sensor (LSe) to block light in the visible part of the electromagnetic spectrum.

14. The device according to claim 8 further comprising an attitude sensor that provides a position and an orientation of the frame with respect to physical world.

15. The device according to claim 8 further comprising a sensor, preferably a camera that provides information about an environment in front of the user.

16. Use of the system according to claim 1 for a monitoring of drowsiness.

17. Use of the device according to claim 8 for a monitoring of drowsiness.
